(19) United States
(12) Patent Application Publication
(10) Pub. No.: US 2008/0294183 A1
(43) Pub. Date: Nov. 27, 2008

(54) Pain-Reducing Device

(76) Inventor: Do Young O, Chungcheongnam-do (KR)

Correspondence Address: AKERMAN SENTERFITT, P.O. BOX 3188, WEST PALM BEACH, FL, 33402-3188, US

(21) Appl. No.: 11/840,562

(22) Filed: Aug. 17, 2007

(30) Foreign Application Priority Data
May 25, 2007 (KR) 10-2007-50708

(51) Int. Cl. A61B 17/34

(52) U.S. Cl. 606/185

(57) ABSTRACT

Disclosed is a pain-reducing device that has a simple structure providing intramuscular stimulation to relieve pain of an affected part. The pain-reducing device includes a butterfly-shaped handle 51, a scalp needle 54 having a canaliculus shape that is provided at a side of the handle 51 and communicates with a syringe insertion unit 53, and a magic silk thread 55 bent at an end thereof and inserted into the scalp needle so that the scalp needle is inserted into an affected part to provide intramuscular stimulation. Since pain is easily and precisely reduced, the effect of the treatment effect can be maximized.

### BACKGROUND OF THE INVENTION

1. Field of the Invention

The present invention relates to a pain-reducing device that has a simple structure providing intramuscular stimulation to relieve pain of an affected part.

2. Description of the Related Art

Intramuscular stimulation (IMS) is known as a treatment method to relieve muscle pain by inserting a needle in the muscle of an affected part. In the treatment method, a fine needle, which is similar to a needle used for acupuncture, is inserted into the muscle and then moved back and forth in the muscle to stimulate the muscle. In this connection, the back-and-forth movement means that the needle is inserted into the muscle, pulled out partially not completely, and then inserted again along the same insertion path. This movement is repeated several times on a plurality spots of the muscle. The muscle of the affected part is shrunken to be rigid. The rigid muscle applies significant pressure to nerve fibers in the muscle or strongly tightens the nerve fibers in the muscle, causing chronic pain. If the shrunken muscle becomes loosened by repeatedly using an intramuscular stimulation treatment, the pain can be relieved. The frequency of treatment depends on how much the muscle has shrunk. A significantly shrunken muscle needs to be frequently treated over a long period of time, but a slightly destroyed muscle does not need to be frequently treated. It is most effective when the intramuscular stimulation treatment is performed on a plurality of spots.

FIG. 5 illustrates a main structure of a motor driven intramuscular stimulation (IMS) needling device that is connected to a controller in order to control a needling speed and depth during the needling.

In order to treat a patient, a syringe body is positioned on the skin of an affected part of the muscle of the patient by an operator. The syringe body has an inlet through which the needle can freely pass. If the controller is operated, the needle starts to move. The needle moves back and forth according to the needling number and depth that are entered beforehand by the controller. Accordingly, the needle is inserted into the skin to stimulate the corresponding muscle and the stimulation is repeated until the needle is removed from the skin.

The needling device is provided with 6 main elements including a linear motor 10, a syringe holder 16, a syringe body 34, a plunger 22, a needle support cap 26, and a needle 32. An inner gear (not shown) is provided in the linear motor 10 to convert a rotation movement of the motor into a reciprocating movement to a shaft 18. The linear motor that performs the above-mentioned operation is manufactured by Philips Technology, Inc., in Cheshire City, Conn., USA. The rotation movement of the motor determines the direction of the linear movement of the needle 32. For example, when the motor 10 rotates clockwise, the shaft 18 moves downward and when the motor 10 rotates counterclockwise, the shaft 18 moves upward. A controller 38 sends both movement signal and direction signal through a wire 36 to the motor 10.

The syringe holder 16 holds the syringe body 34. The syringe holder 16 is fixed to a substrate 12 of the motor 10 by two holding screws 14 and acts as a mechanical working part that is used to form a housing for a syringe substrate 20 connected to the syringe body 34. Preferably, the syringe holder 16 is made of a material which is sold under the trademark “Plexiglas”, and may be mechanically worked so that the syringe body 34 is easily fastened and unfastened in respects to the syringe holder 16, for example, based on a twist-lock mechanism. The syringe body 34 is made of a transparent polymer material so that movement of the needle 32 can be seen from the outside of the syringe body 34. For example, a commercial disposable syringe having a volume of 10 cc may be used as the syringe body 34.

The plunger 22 is produced as a mechanical working part that is made of a plastic material, and functions to connect the motor 10 and the needle 32 to each other. As shown in FIG. 5, the plunger 22 is attached to the motor shaft 18 at an end thereof by using a set screw 24. A holding pin 28 is provided at the other end of the plunger 22 to hold a needle holding cap 26 according to the twist-lock mechanism. Preferably, the needle holding cap 26 is produced as a mechanical working part and functions to firmly fix the needle 32 so that the needle 32 is aligned along the axis of the shaft 18 during treatment of a patient. A twist lock slot is formed in an upper part of the cap 26 to quickly replace the needle therethrough. A mechanically worked hole is formed in a lower part of the cap 26 to receive a head 30 of the needle 32 therethrough. Preferably, the needle 32 has a very small diameter in order to be effectively inserted into the skin and the muscular tissue, similar to the needle used for acupuncture. Preferably, the needle 32 may be made of flexible stainless steel and a surgically acceptable material. A plastic head 30 is provided to desirably support the needle 32.

However, the above-mentioned known intramuscular stimulation needling device is problematic in that since the device has a complicated structure including many elements, production cost is high.

### SUMMARY OF THE INVENTION

Accordingly, the present invention has been made in consideration of the above disadvantages in the related arts, and an object of the present invention is to provide a pain-reducing device that has a simple structure providing intramuscular stimulation to relieve a pain of an affected part by using a syringe method. Thus, the pain-reducing device is capable of being economically and easily used by those who skilled in the art.

In order to achieve the above object, the present invention provides a pain-reducing device including a butterfly-shaped handle, a scalp needle having a canaliculus shape which is provided at a side of the handle and communicates with a syringe insertion unit, and a magic silk that is inserted into the scalp needle to be provided in a muscle when the scalp needle is inserted into an affected part to provide intramuscular stimulation.

### BRIEF DESCRIPTION OF THE DRAWINGS

FIG. 1 is an exploded view of a portion of a pain-reducing device according to a first embodiment of the present invention;

FIG. 2 is a perspective view of the pain-reducing device according to the first embodiment of the present invention;

FIG. 3 is an exploded view of a portion of a pain-reducing device according to a second embodiment of the present invention;

FIG. 4 is a perspective view of the pain-reducing device according to the second embodiment of the present invention; and

FIG. 5 is a side view of a known syringe for intramuscular stimulation.

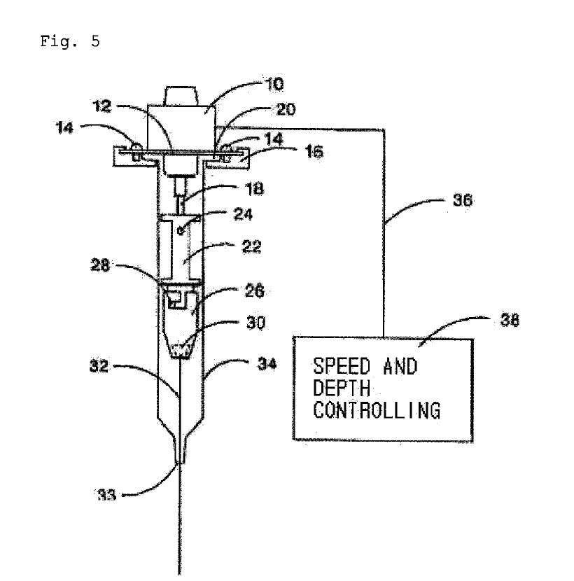

### DESCRIPTION OF THE PREFERRED EMBODIMENTS

Hereinafter, first and second embodiments of the present invention will be described in detail with reference to the accompanying drawings.

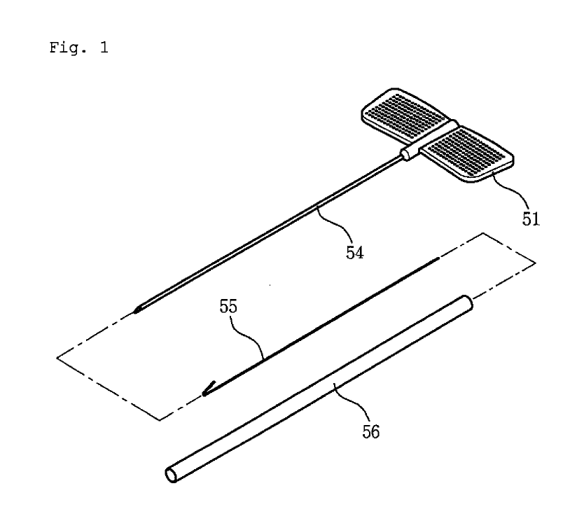

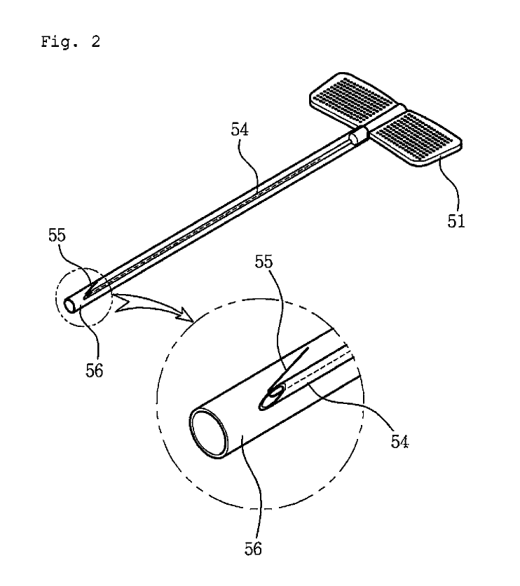

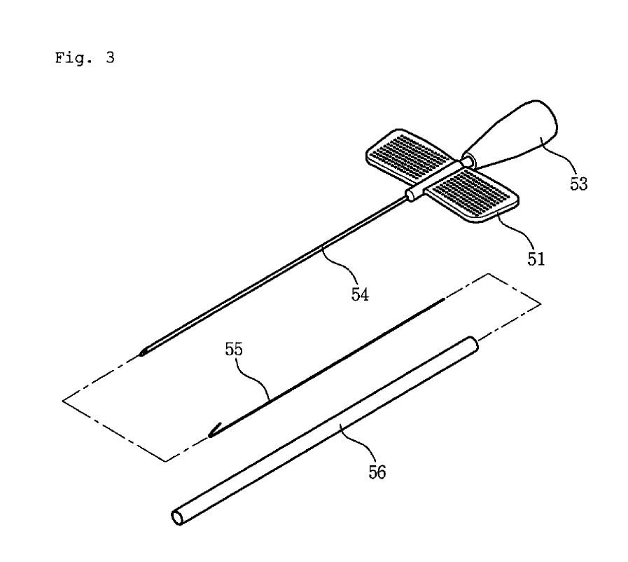

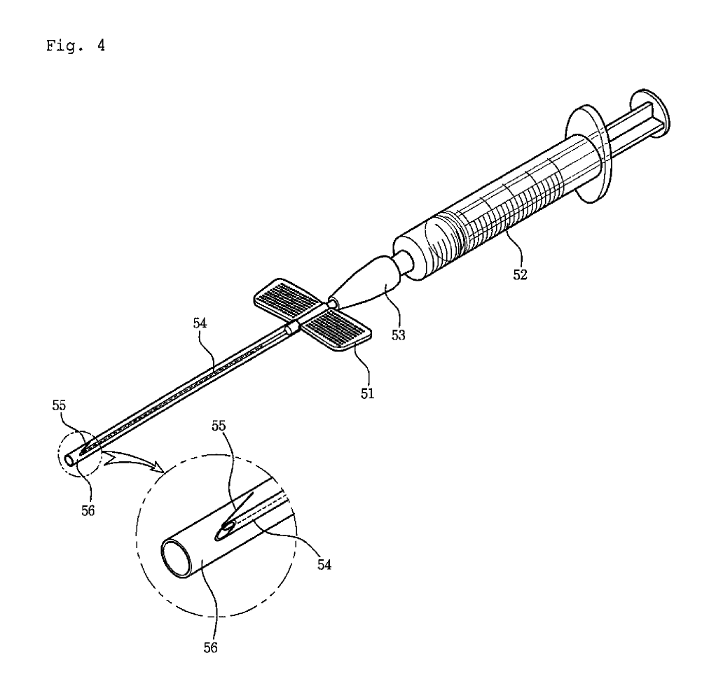

FIG. 1 is an exploded view of a portion of a pain-reducing device according to a first embodiment of the present invention, FIG. 2 is a perspective view of the pain-reducing device according to the first embodiment of the present invention, FIG. 3 is an exploded view of a portion of a pain-reducing device according to a second embodiment of the present invention, and FIG. 4 is a perspective view of the pain-reducing device according to the second embodiment of the present invention.

As shown in FIGS. 1 and 2, a pain-reducing device according to a first embodiment of the present invention includes a butterfly-shaped handle 51, a scalp needle 54 having a canaliculus shape that is provided at a side of the handle 51 and communicates with the syringe insertion unit 53, and a magic silk thread 55 bent at an end thereof and inserted into the scalp needle so that the scalp needle is inserted into an affected part to provide intramuscular stimulation.

As shown in FIGS. 3 and 4, the pain-reducing device according to a second embodiment of the present invention further includes a syringe insertion unit 53 that is provided at the other side of the handle 51 opposite to the scalp needle 54 and formed of a cylinder gradually expanding in diameter so as to insert a syringe 52.

A needle protection tube 56 that is made of plastics is provided to surround the scalp needle 54, and fitted to a protrusion on the side of the handle 51 at an end thereof.

The magic silk is used as an absorbable suture and made of collagen that is extracted from healthy mammals as a raw material. The magic silk is absorbed on tissue during treatment of tissue. An absorption time thereof depends on a physical condition, and if infection occurs or a nutritive condition is good, rapid absorption is ensured. This is because the blood circulation in adipose tissue and muscular tissue is desirable.

The magic silk 55 is spontaneously absorbed to prevent a reaction from occurring due to foreign material, in particular, is capable of being used even though inflammation occurs. Extracted from the submucosa in the internal organs of sheep or the serous membrane tissue in the internal organs of cows, the magic silk consists of protein as a main component (protein concentration: 98%). The magic silk slowly is degraded by the enzyme in humans, causing the suture to be absorbed in the interior body tissue. In particular, the magic silk is very quickly absorbed on the serous membrane tissue or the mucous tissue, but very slowly absorbed on the subcutaneous fat tissue.

During the pain treatment, if an operator holds the handle 51 and inserts the scalp needle 54, in which the magic silk 55 is inserted, into a target affected part to a predetermined depth, the magic silk 55 is inserted into the dermatic tissue and absorbed therein to easily and precisely relieve the pain.

Like the theory suggested by Dr. Gunn, it is possible to treat pain and the symptoms of an internal disease by using the pain-reducing device. In addition, a fine current of energy, which is most suitable to humans, can be continuously generated by insertion of the magic silk, so as to achieve a favorable treatment effect, which is found in the bioelectric energy theory proposed by Robert Becker, an American orthopedist. The book of Dr. Jarnet. G. Travell, which is entitled “Fascia pain and Dysfunction”, discloses use of an appropriate syringe method in respects to a trigger point and a motor point.

The pain-reducing device of the present invention and a known acupuncture treatment are compared and described in the following Table.

| Pain-reducing device of the | |
|---|---|
| present invention | Known acupuncture treatment |
| A medical diagnosis is required | A medical diagnosis is not required |
| | because spots on the body suitable |
| | for acupuncture are used |
| A medical examination is | A medical examination is not |
| necessary | necessary |
| Portions to be treated such as a | Portions to be treated such as spots |
| trigger point and a motor point | on the body suitable for acupuncture |
| are determined according to the | are not determined by a scientific |
| results of the scientific | method but based on Chinese |
| examination | philosophy |
| Anatomic and neurophysiologic | Anatomic and neurophysiologic |
| knowledge is necessary | knowledge is not necessary |
| Immediate subjective and objective | Immediate objective effects cannot |
| effects are obtained | be expected |

According to the pain-reducing device of the present invention, since the handle is provided at the scalp needle to insert the magic silk into the dermatic tissue, it is possible to easily and precisely reduce pain and the effects of the treatment can be maximized.

The pain-reducing device of the present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

What is claimed is:

1. A pain-reducing device comprising:
a butterfly-shaped handle;
a scalp needle that is provided at a side of the handle and has a canaliculus shape; and
a magic silk thread bent at an end thereof and inserted into the scalp needle so that the scalp needle is inserted into an affected part to provide intramuscular stimulation.

2. The pain-reducing device according to claim 1, further comprising:
a syringe insertion unit that is provided at the other side of the handle opposite to the scalp needle and formed of a cylinder gradually expanding in diameter so as to insert a syringe.

3. The pain-reducing device according to claim 1, further comprising:
a needle protection tube that is fitted to a protrusion on the side of the handle at an end thereof to surround the scalp needle and made of a plastic material.
